(19) United States
(12) Patent Application Publication
(10) Pub. No.: US 2025/0195047 A1
(43) Pub. Date: Jun. 19, 2025

(54) BONE BIOPSY ASSEMBLY WITH LOST MOTION RECEIVER

(71) Applicant: University of Kentucky Research Foundation, Lexington, KY (US)

(72) Inventors: Clay Larkin, Lexington, KY (US); Madhumathi Rao, Lexington, KY (US); Florence Lima, Lexington, KY (US)

(21) Appl. No.: 18/849,112

(22) PCT Filed: Mar. 20, 2023
(86) PCT No.: PCT/US2023/064707

§ 371 Date: Sep. 20, 2024

Related U.S. Application Data
(60) Provisional application No. 63/323,012, filed on Mar. 23, 2022.

(51) Int. Cl. A61B 10/02

(52) U.S. Cl. CPC A61B 10/025

(57) ABSTRACT

A bone biopsy assembly, includes (a) a lost motion receiver, (b) a trephine needle, including an elongated shaft having a first end, a second end and a lumen extending from the first end to the second end, and (c) a stylet, received for sliding movement within the lumen and the lost motion receiver. A method of procuring a bone core sample from a bone, includes steps of (a) engaging a pointed tip of a stylet into the bone to seat a cutting edge of a trephine needle in the bone, (b) unlocking the stylet to allow free sliding movement of the stylet within a lumen of the trephine needle and (c) driving the trephine needle into the bone to procure the bone core sample within the lumen of the trephine needle.

### TECHNICAL FIELD

This document relates generally to a bone biopsy system as well as to a related method of procuring a bone core sample from a bone of a patient.

### BACKGROUND

A bone biopsy is a medical procedure where a core of bone is obtained from the iliac crest of a patient for diagnostic purposes. It is the gold standard for diagnosing and guiding treatment for a wide range of metabolic bone disorders.

There is currently no dedicated bone biopsy needle on the market. The centers that perform this procedure use and adapt bone marrow biopsy needles or power-driven handheld systems generally used for bone access by orthopedic surgeons.

This document relates to a novel (a) bone biopsy assembly, (b) bone biopsy system and (c) related method of procuring a bone core sample from a bone of a patient all dedicated to the bone biopsy procedure. The design for the needle is specific to the bone biopsy procedure and minimizes tissue injury and complications to the patient. In addition, the bone biopsy system, incorporating that bone biopsy needle, and the related method of procuring a bone core sample from the bone of a patient address ease of use for the operator.

### SUMMARY

In accordance with the purposes and benefits described herein, a new and improved bone biopsy assembly and method of procuring a bone core sample from a bone are provided. The bone biopsy assembly comprises, consists of or consists essentially of: (a) a lost motion receiver having a proximal end and a distal end, (b) a trephine needle including an elongated shaft having a first end, a second end and a lumen extending from the first end to the second end and (c) a stylet received for sliding movement within the lumen and the lost motion receiver.

In one or more of the many possible embodiments, the bone biopsy assembly further includes a drive connector at the proximal end of the lost motion receiver and a cutting edge at the first end of the trephine needle.

In one or more of the many possible embodiments of the bone biopsy assembly, the bone biopsy assembly further includes a coupler releasably securing the lost motion receiver to the trephine needle. The coupler may further include a male connector at the second end of the trephine needle and a cooperating female connector at the distal end of the lost motion receiver. Still further, the coupler may further include a locking ring carried on the lost motion receiver and a cooperating locking boss carried on the trephine needle.

In one or more of the many possible embodiments of the bone biopsy assembly, the stylet includes an elongated body having a pointed tip at a first terminus and a head at a second terminus.

In one or more of the many possible embodiments of the bone biopsy assembly, the bone biopsy assembly further includes a stylet seat at the second end of the trephine needle. The stylet seat is adapted for receiving and holding the head of the stylet when the stylet is positioned within the trephine needle in a trephine needle seating position with the pointed tip projecting beyond the cutting edge. In at least some embodiments, that stylet seat may be provided in the male connector.

In one or more of the many possible embodiments of the bone biopsy assembly, the stylet seat may be in the form of an acircular socket and the head includes an acircular cross section whereby when the stylet is in the trephine needle seating position, the head is received in the acircular socket and the stylet is locked for rotation with the trephine needle.

In one or more of the many possible embodiments of the bone biopsy assembly, the bone biopsy assembly further includes a removable stop that is engaged in an opening in the lost motion receiver to secure the stylet in the trephine needle seating position when the trephine needle is being seated in the bone prior to boring.

In one or more of the many possible embodiments of the bone biopsy assembly, the lost motion receiver includes an outer wall outlining a lost motion chamber adapted to accommodate sliding movement of the stylet during bone boring with the bone biopsy assembly. Further, the outer wall may include at least one window allowing one to view the stylet as it slides in the lost motion chamber of the lost motion receiver.

In at least one or more of the many possible embodiments of the bone biopsy assembly, the bone biopsy assembly further includes indicia on (a) the trephine needle, (b) the lost motion receiver or (c) the trephine needle and the lost motion receiver whereby one is able to visually monitor in real time a depth of the bone biopsy during bone boring.

In one or more of the many possible embodiments of the bone biopsy assembly, the bone biopsy assembly further includes the stylet seat at the second end of the trephine needle. The stylet seat is adapted for receiving and holding the head of the stylet when the stylet is positioned within the trephine needle in a trephine needle seating position with the pointed tip projecting beyond the cutting edge. That stylet seat may take the form of an acircular socket and the head includes an acircular cross section whereby when the stylet is in the trephine needle seating position, the head is received in the acircular socket and the stylet is locked for rotation with the trephine needle.

A removable stop may be engaged in an opening in the lost motion receiver to secure the stylet in the trephine needle seating position when the trephine needle is being seated in the bone prior to boring.

In accordance with yet another aspect, a new and improved method is provided for procuring a bone core sample from a bone. That method comprises, consists of or consists essentially of the steps of: (a) engaging a pointed tip of a stylet into the bone to seat a cutting edge of a trephine needle in the bone, (b) unlocking the stylet to allow free sliding movement of the stylet within a lumen of the trephine needle and (c) driving the trephine needle into the bone to procure the bone core sample within the lumen of the trephine needle while allowing the stylet to slide through the lumen of the trephine needle in front of the bone core sample.

In one or more of the many possible embodiments of the method, the method includes the step of removing the trephine needle from the bone. In one or more of the many possible embodiments of the method, the method includes the step of removing the stylet from the lumen of the trephine needle. In one or more of the many possible embodiments of the method, the method includes the step of pushing the bone core sample out of the lumen of the trephine needle.

In accordance with yet another aspect, a new and improved method of procuring a bone core sample from a bone, comprises, consists of or consists essentially of:
connecting a driver to a bone biopsy assembly including (a) a lost motion receiver, (b) a trephine needle and (c) a stylet received for sliding movement within the lost motion receiver and a lumen of the trephine needle;
engaging a pointed tip of the stylet into the bone to seat a cutting edge of the trephine needle in the bone;
unlocking the stylet to allow free sliding movement of the stylet within the lumen of the trephine needle and the lost motion receiver; and
driving the trephine needle into the bone to procure the bone core sample within the lumen of the trephine needle while allowing the stylet to slide through the lumen of the trephine needle in front of the bone core sample.

Such a method may further include the step of (a) removing the trephine needle from the bone, (b) removing the stylet from the lumen of the trephine needle and/or (c) pushing the bone core sample out of the lumen of the trephine needle. Still further, the method may include the step of only connecting the driver to the bone biopsy assembly once during the entire process of procuring of the bone core sample.

In the following description, there are shown and described several embodiments of the novel (a) bone biopsy assembly, (b) the bone biopsy system and (c) the related method of procuring a bone core sample from a bone of a patient. As it should be realized, the bone biopsy assembly, bone biopsy system and related method are capable of other, different embodiments and their several details are capable of modification in various, obvious aspects all without departing from the bone biopsy assembly, the bone biopsy system and method as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the novel bone biopsy assembly, the bone biopsy system and related method of procuring a bone core sample from a bone of a patient and together with the description serve to explain certain principles thereof.

FIG. 1 is a side elevational view of the bone biopsy system in a disassembled state, including the powered driver, the stylet, the trephine needle, the lost motion receiver, the stop, the locking ring and the plunger.

FIG. 2A is a detailed side elevational view of the trephine needle. FIGS. 2B and 2C are respective detailed distal end and proximal end views of the trephine needle illustrated in FIG. 2A.

FIG. 3A is a detailed side elevational view of the stylet. FIGS. 3B and 3C are respective detailed distal end and proximal end views of the stylet illustrated in FIG. 3A.

FIG. 4A is a detailed side elevational view of the lost motion receiver. FIGS. 4B and 4C are respective detailed distal end and proximal end views of the lost motion receiver illustrated in FIG. 4A.

FIG. 5 is a detailed cross-sectional view of the locking ring.

FIG. 6 is a detailed side elevational view of the removable stop.

FIG. 7 is a detailed longitudinal cross section illustrating the stylet fully seated in the trephine needle.

FIG. 8 is a side elevational view of the fully assembled device.

FIG. 9 is a longitudinal cross section illustrating the fully assembled device of FIG. 8.

FIGS. 10A-10H illustrate the new method of procuring a bone core sample from a bone of a patient.

FIG. 10A shows the connecting of the chuck, on the driver, to the hex shaft on the lost motion receiver.

FIG. 10B shows the pointed tip of the stylet engaging in the bone and the seating of a cutting edge of the trephine needle in the bone to provide a stable point for taking the bone sample with the trephine needle.

FIG. 10C shows the removing of the stop from the aperture in the lost motion receiver.

FIG. 10D is a longitudinal cross section illustrating the top of the stylet at the “20” mark on the lost motion receiver and a bone sample in the end of the needle.

FIG. 10E shows the removing of the lock ring.

FIG. 10F shows the removing of the lost motion receiver from the proximal end of the trephine needle.

FIG. 10G shows the removing of the stylet from the trephine needle.

FIG. 10H shows the plunger inserted in the distal end of the trephine needle and used to push the bone sample from the proximal end of the trephine needle.

### DETAILED DESCRIPTION

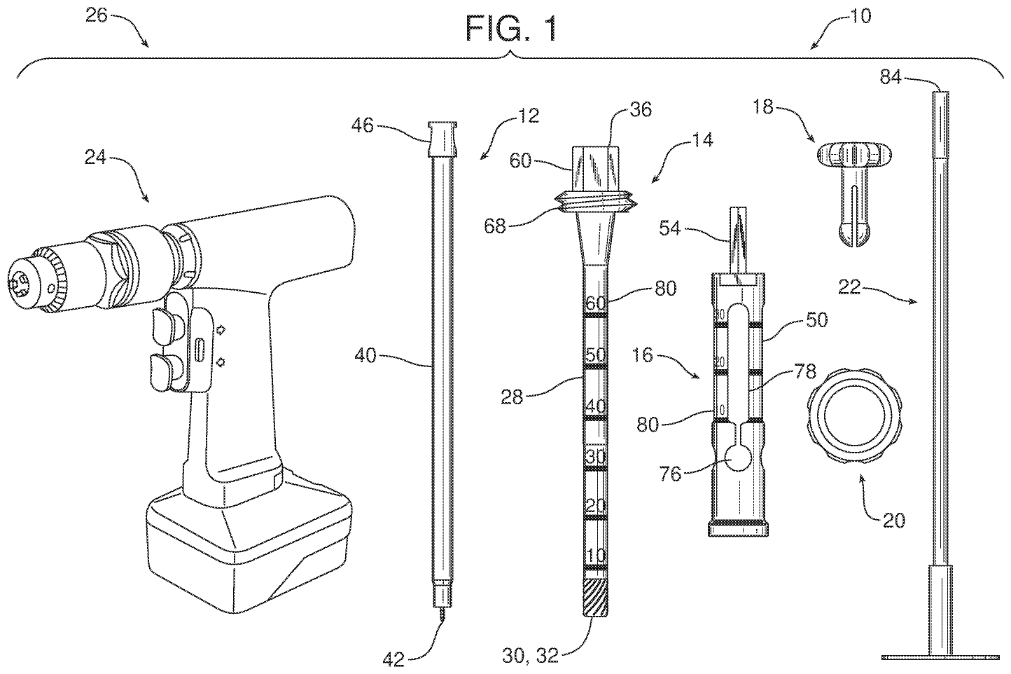

Reference is now made to FIG. 1 which illustrates the various components of the novel bone biopsy needle assembly 10 in a disassembled state or condition. As shown, the bone biopsy needle assembly 10 generally includes a stylet 12, a trephine needle 14, a lost motion receiver 16, a removable stop 18, a locking ring 20 and a push rod or plunger 22. FIG. 1 also shows a powered driver 24. Together, the bone biopsy needle assembly 10 and the powered driver 24 may be considered to comprise a novel bone biopsy system 26. In other embodiments, a manually operated driver may be used in place of the illustrated powered driver 24.

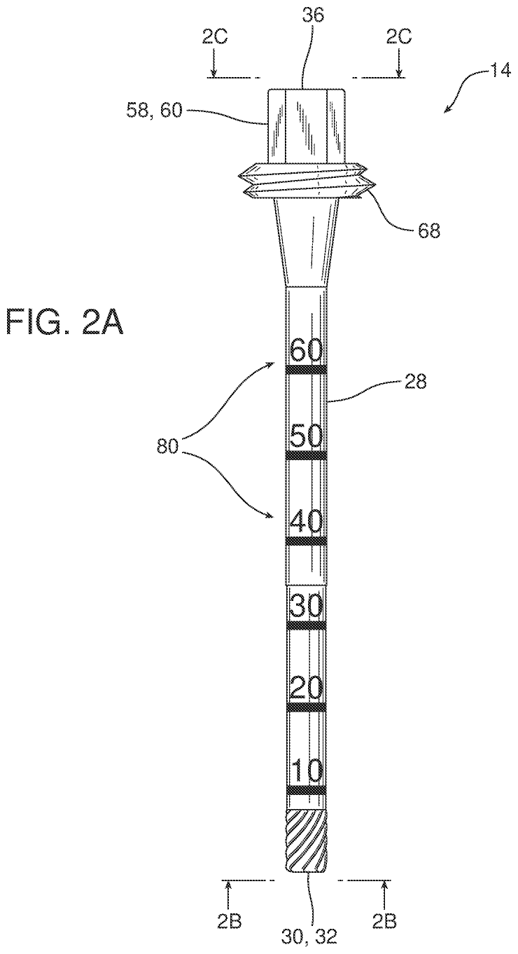

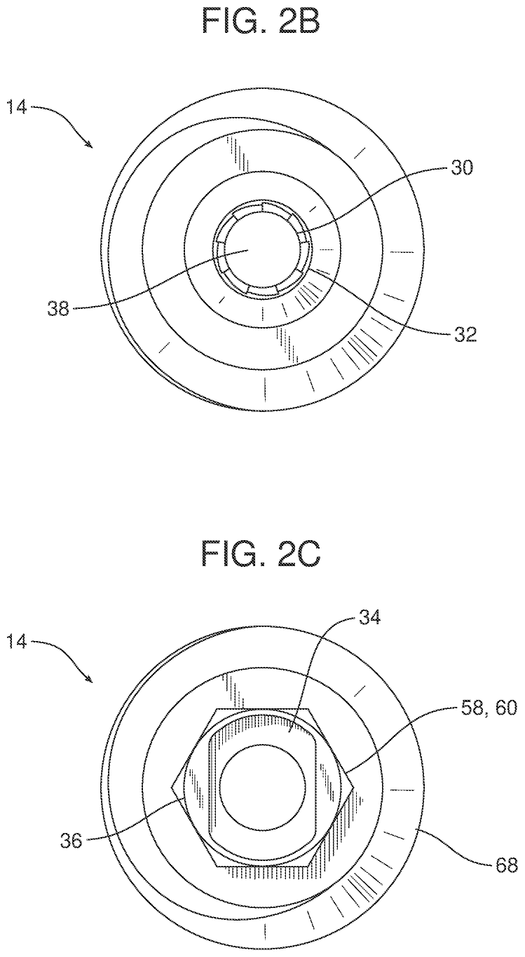

The trephine needle 14 includes an elongated shaft 28 having (a) a cutting edge 30 at a first end 32, (b) a stylet seat 34 at second end 36 and (c) a lumen 38 extending from the first end to the second end. See FIGS. 2A-2C, 7 and 8.

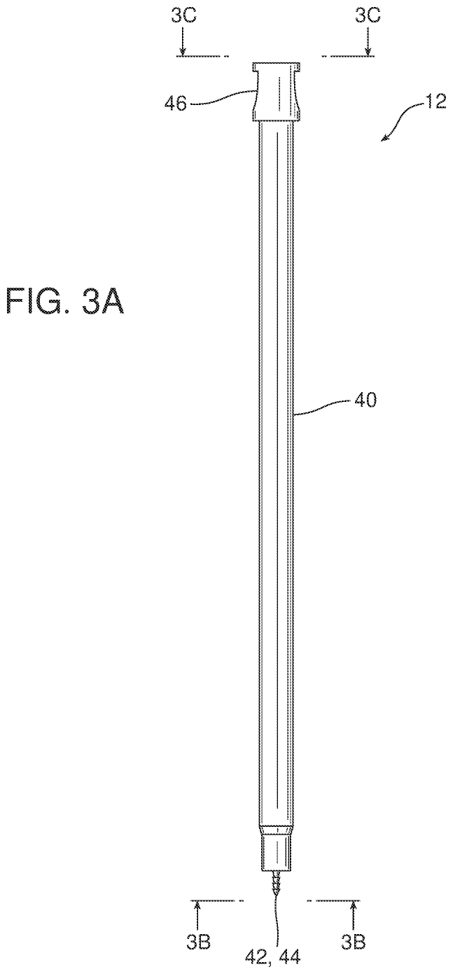

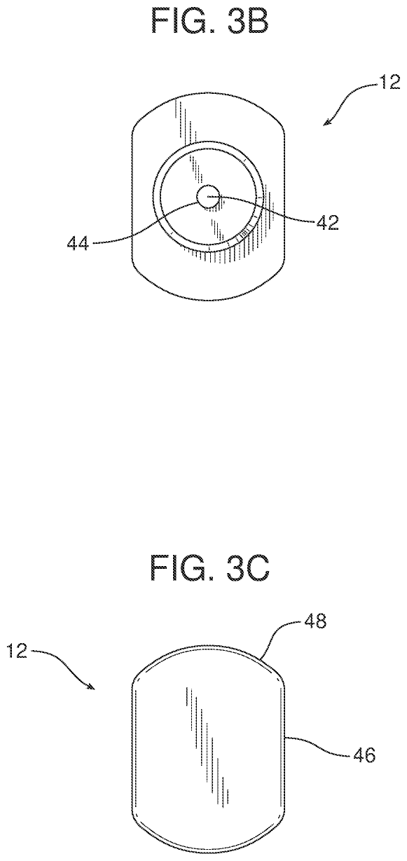

The stylet 12 includes an elongated body 40 having a pointed tip 42 at a first terminus 44 and a head 46 at a second terminus 48. See FIGS. 3A-3C, 7 and 8.

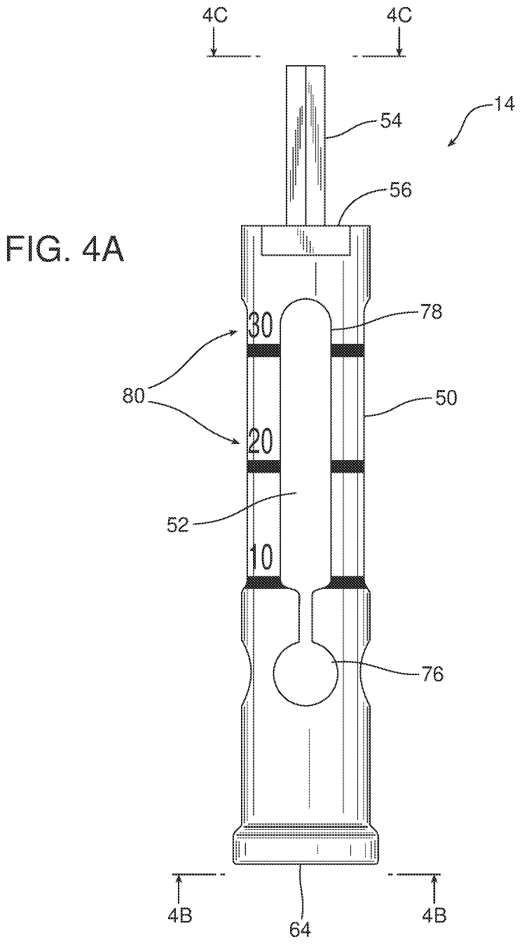

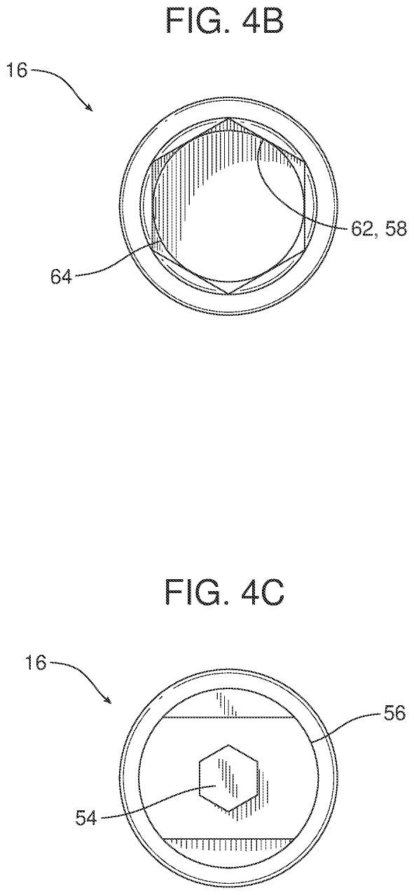

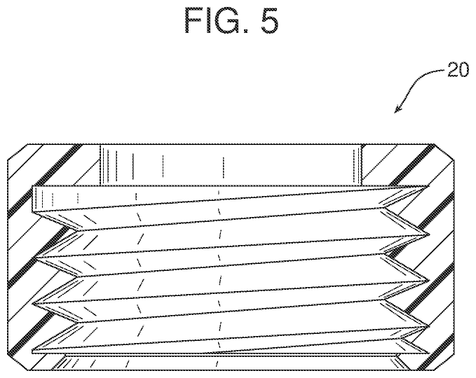

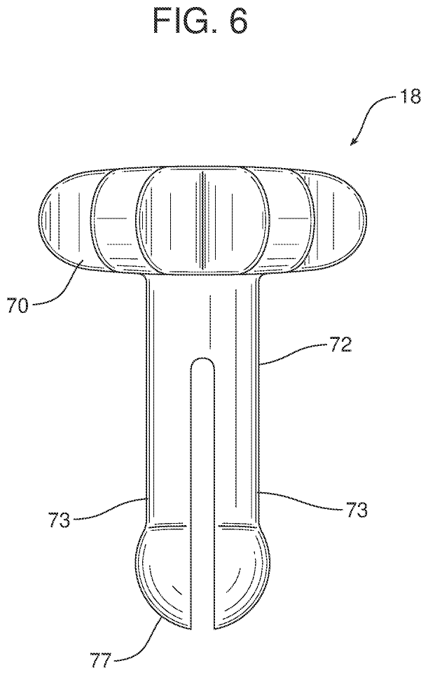

As best illustrated in FIGS. 4A-4C, the lost motion receiver 16 includes an outer wall 50 that outlines and defines a lost motion chamber 52. The lost motion chamber 52 is adapted to accommodate sliding movement of the stylet 12 during bone boring with the bone biopsy assembly 10 in a manner described below. A drive connector 54, in the form of a shank with a hexagonal cross section, is provided at the proximal end 56 of the lost motion receiver 16. This allows for connection of the bone biopsy assembly 10 to the powered driver 24 in a manner described below.

The bone biopsy assembly 10 also includes a coupler 58 that releasably secures the lost motion receiver 16 to the trephine needle 14. In the illustrated embodiment, the coupler 58 includes (a) a male connector 60 at the second end 36 of the trephine needle 14, (b) a cooperating female connector 62 at the distal end 64 of the lost motion receiver 16, (c) a locking ring 20 carried on the lost motion receiver 16 and (d) a cooperating locking boss 68 carried on the trephine needle.

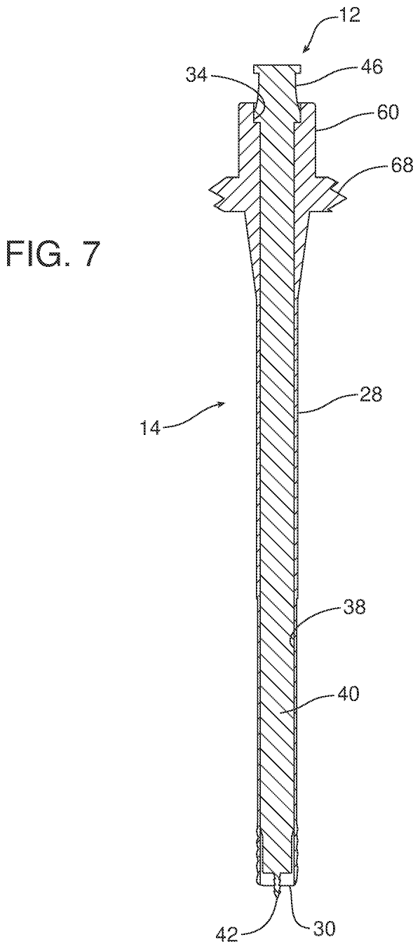

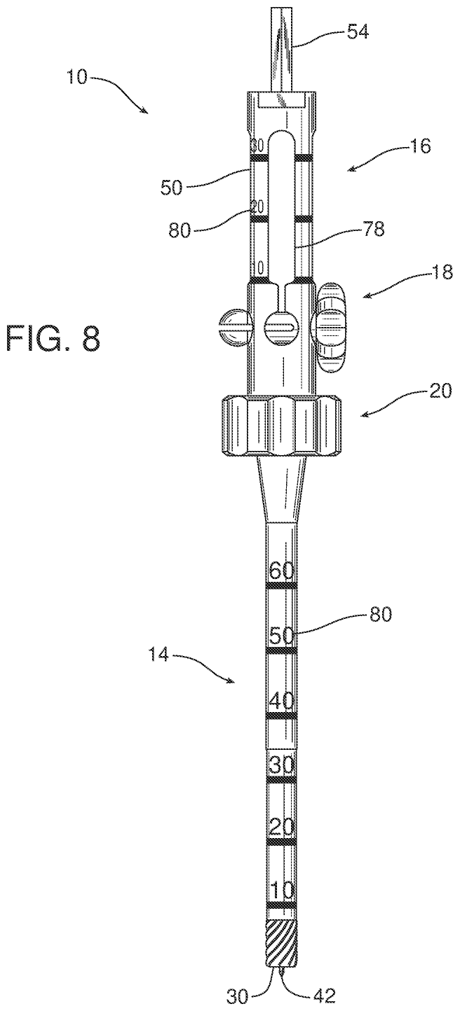

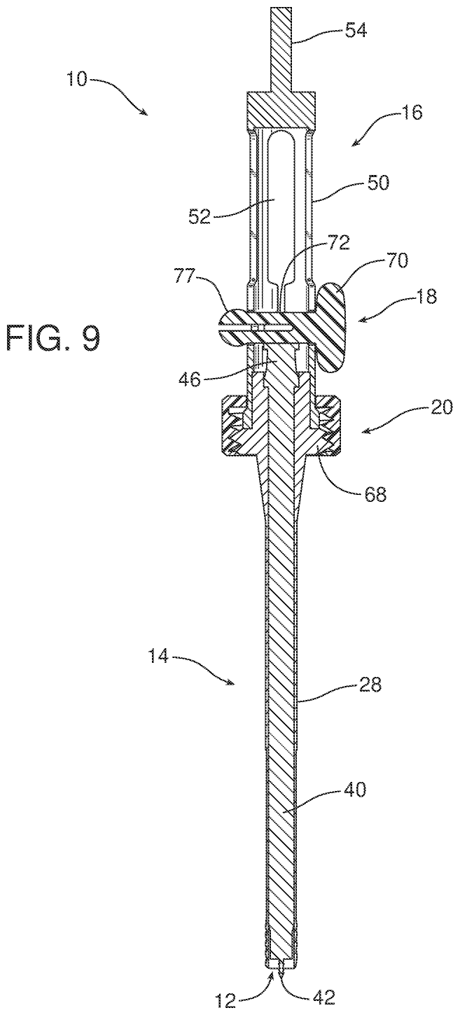

In the illustrated embodiment, the stylet seat 34 is provided in the male connector 60. The stylet seat 34 takes the form of an acircular socket and the head 46 includes an acircular cross section whereby when the stylet 12 is in the trephine needle seating position, the head is received in and nests with the stylet seat/acircular socket 34 with the pointed tip 42 projecting slightly from the cutting edge 30. See FIGS. 7 and 8.

As previously noted, the lost motion receiver 16 includes an outer wall 50 that outlines and defines a lost motion chamber 52. The lost motion chamber 52 is adapted to accommodate sliding movement of the stylet 12 with respect to the trephine needle 14 and the lost motion receiver 16 during bone boring with the bone biopsy assembly 10 in a manner described below.

The removable stop 18 may be engaged in an opening 76 in the outer wall 50 of the lost motion receiver 16 in order to secure the stylet 12 in the trephine needle seating position when the trephine needle 14 is being seated in the bone, from which a bone sample is to be taken, prior to boring. More specifically, the removable stop 18 includes a head 70 and a split shank 72 having two resilient legs 73. One holds the stop 18 by the head 70 and pushes the shank 72 through the opening 76. As this is done, the resilient legs 73 squeeze together so that the enlarged end 77 is able to pass through the opening 76. Once through, the legs 73 spring back outward and the enlarged end then tends to hold the stop in the opening 76 securing the stylet 12 in the trephine needle seating position. At least one window 78, provided in the outer wall 50 of the lost motion receiver 16 allows one to view the position of the stylet 12 as it slides in the lost motion chamber 52 during bone boring.

Indicia 80 may be provided to allow one to monitor the depth of the bone biopsy in real time during the bone boring procedure. That indicia 80 may be provided on the trephine needle 14, the lost motion receiver 16 or on both of the trephine needle and the lost motion receiver. See FIGS. 4A and 8.

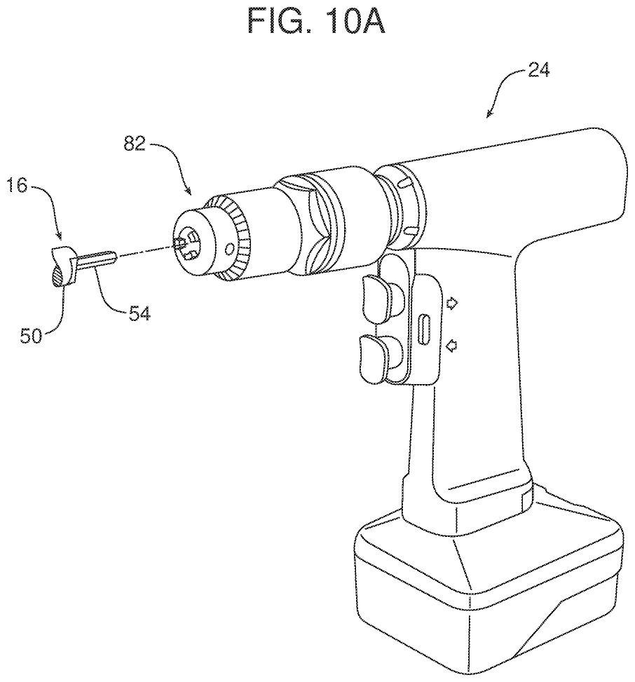

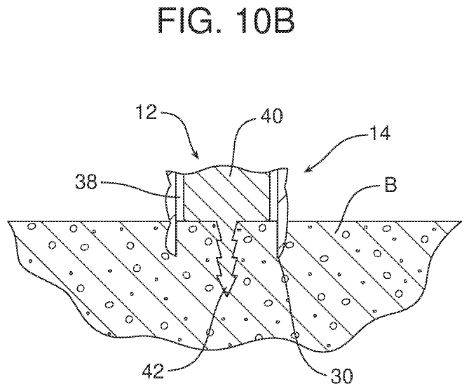

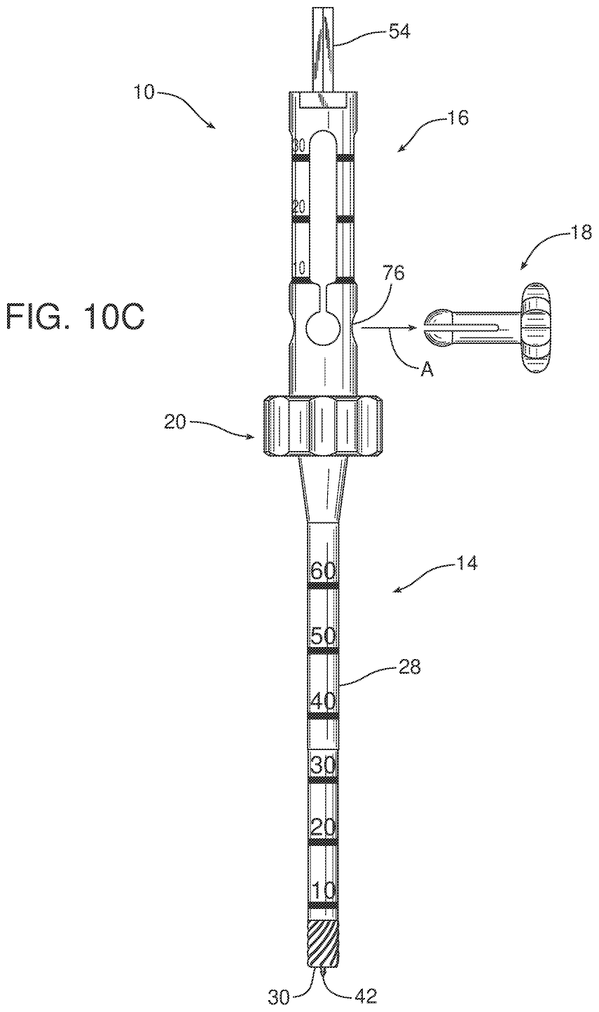

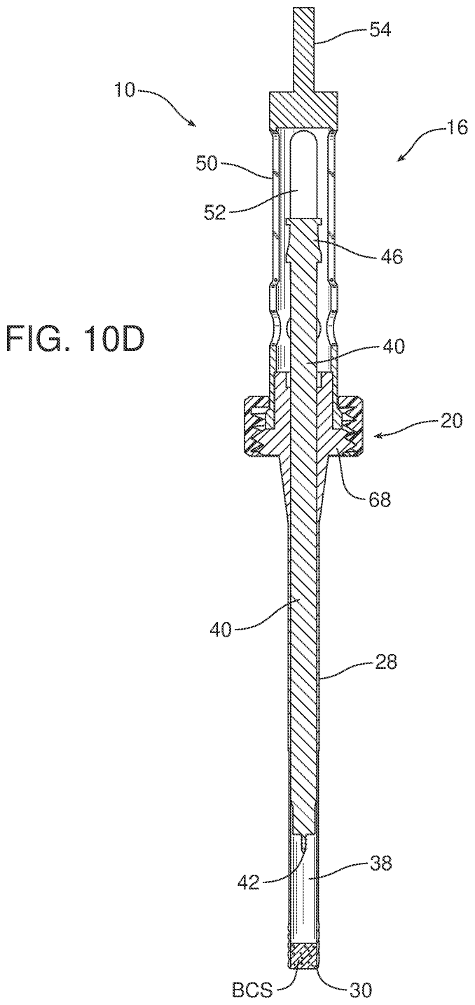

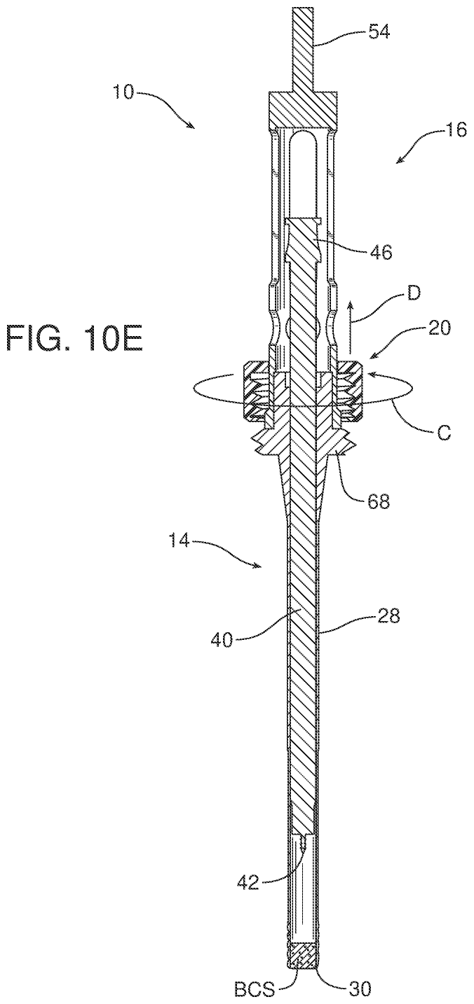

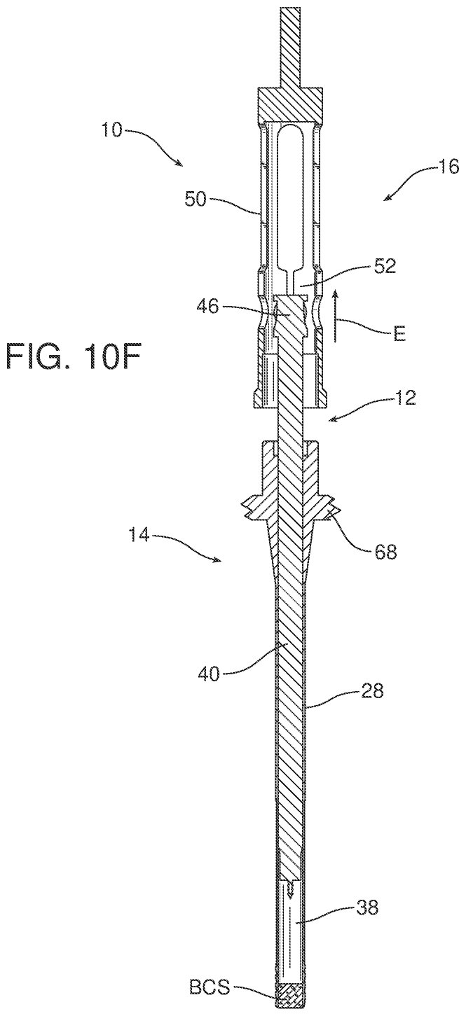

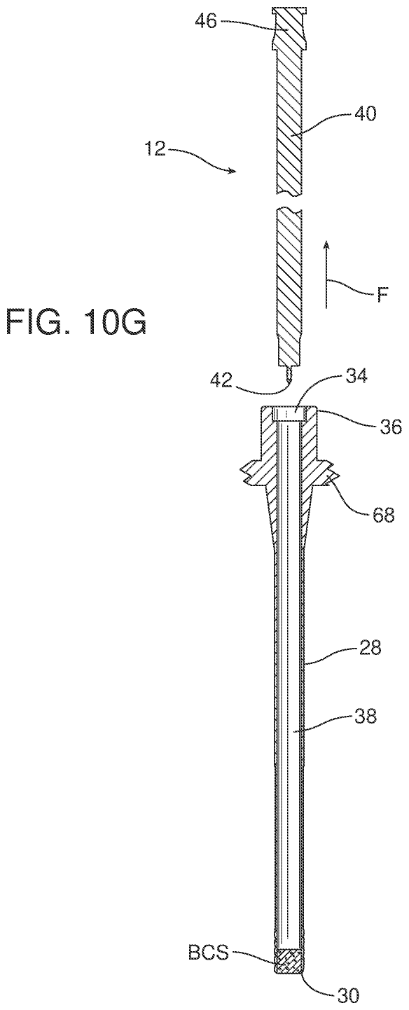

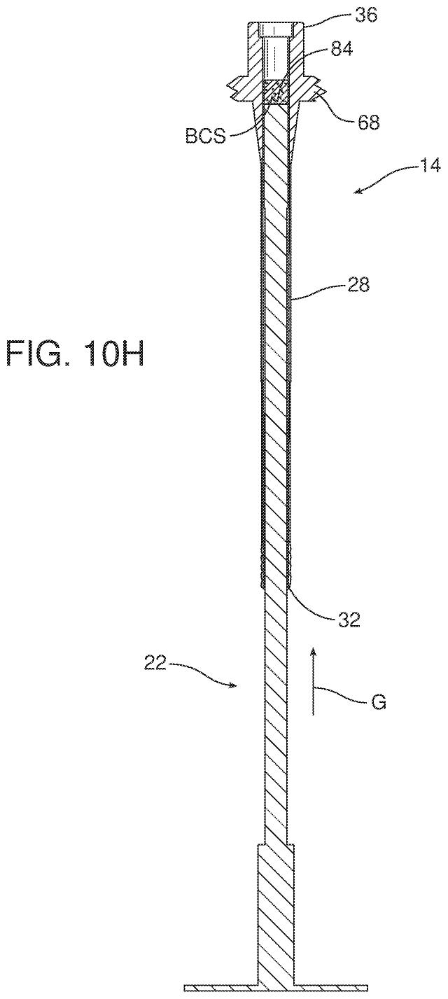

The bone biopsy assembly 10 may be used in a method of procuring a bone core sample from a bone B. That method may include the step of connecting a powered driver 24, of a type known in the art, to a bone biopsy assembly 10 including (a) a lost motion receiver 16, (b) a trephine needle 14 and (c) a stylet 12 received for sliding movement with the lost motion receiver and a lumen 38 of the trephine needle. As illustrated in FIG. 10A, this may be done by tightening a chuck 82, on the driver 24, to the drive connector (hex shaft) 54 on the lost motion receiver 16.

Next is the engaging of the pointed tip 42 of the stylet 12 into the bone B to seat the cutting edge 30 of the trephine needle 14 in the bone B. See FIG. 10B. The pointed tip 42 provides stability, effectively preventing the needle 14 from wondering during the initial stages of the bone boring. This is then followed by the step of unlocking the stylet 12 to allow free sliding movement of the stylet within the lumen 38 of the trephine needle 14 and the lost motion chamber 52 of the lost motion receiver 16. This is done by removing the removable stop 18 from the opening 78 in the outer wall 50 of the lost motion receiver 16. See Action Arrow A in FIG. 10C.

The next step is the driving the trephine needle 14 into the bone B to procure the bone core sample BCS within the lumen 38 of the trephine needle 14 while allowing the stylet 12 to slide through the lumen of the trephine needle in front of the bone core sample BCS. As illustrated in FIG. 10D, a bone core sample of 20 mm has been procured.

Next the trephine needle 14 is carefully removed/withdrawn from the bone B while maintaining the bone core sample BCS in the lumen 38 of the trephine needle. The stylet 12 is then removed from the trephine needle. This is done by loosening the chuck 82 and disconnecting the driver 24 from the bone biopsy assembly 10. The locking ring 20 is then removed by unscrewing the locking ring from the locking boss 68 (see Action Arrows C and D in FIG. 10E). This allows one to remove the lost motion receiver 16 from the second end 36 of the trephine needle 14 (see Action Arrow E in FIG. 10F). Next is the removing of the stylet 12 from the trephine needle 14. More particularly, the stylet 12 may be carefully withdrawn from the second end 36 of the trephine needle 14 by grasping the head 46 and pulling (see Action Arrow F in FIG. 10G).

As illustrated in FIG. 10H, this allows one to carefully insert the plunger 22 into the first end 32 of the trephine needle 14 so that the end 84 of the plunger comes into engagement with the bone core sample BCS within the lumen 38. By applying gentle pressure (see Action Arrow G in FIG. 10H) it is then possible to push the bone core sample BCS out of the second end 36 of the trephine needle 14. The lumen 38 of the trephine needle 14 may taper slightly outward in the direction of the second end 36 to aid in the removal of the bone core sample BCS. That bone core sample BCS may then be biopsied. Advantageously, it should be appreciated that it is only necessary to connect the powered driver 24 to the bone biopsy assembly 10 one time during the entire process of procuring the bone sample. This is a very convenient aspect and time conserving advantage of the method.

This disclosure may be said to relate to the following items.
1. A bone biopsy assembly, comprising:
   a lost motion receiver having a proximal end and a distal end;
   a trephine needle including an elongated shaft having a first end, a second end and a lumen extending from the first end to the second end; and
   a stylet received for sliding movement within the lumen and the lost motion receiver.
2. The bone biopsy assembly of item 1, further including a drive connector at the proximal end of the lost motion receiver and a cutting edge at the first end of the trephine needle.
3. The bone biopsy assembly of item 2, further including a coupler releasably securing the lost motion receiver to the trephine needle.
4. The bone biopsy assembly of item 3, wherein the coupler includes a male connector at the second end of the trephine needle and a cooperating female connector at the distal end of the lost motion receiver.
5. The bone biopsy assembly of item 4, wherein the coupler further includes a locking ring carried on the lost motion receiver and a cooperating locking boss carried on the trephine needle.
6. The bone biopsy assembly of item 5, wherein the stylet includes an elongated body having a pointed tip at a first terminus and a head at a second terminus.
7. The bone biopsy assembly of item 6, further including a stylet seat at the second end of the trephine needle adapted for receiving and holding said head of said stylet when the stylet is positioned within the trephine needle in a trephine needle seating position with the pointed tip projecting beyond the cutting edge.
8. The bone biopsy assembly of item 7, wherein the stylet seat is provided in the male connector.
9. The bone biopsy assembly of item 8, wherein the stylet seat is in the form of an acircular socket and the head includes an acircular cross section whereby when the stylet is in the trephine needle seating position, the head is received in the acircular socket and the stylet is locked for rotation with the trephine needle.
10. The bone biopsy assembly of item 9, further including a removable stop that is engaged in an opening in the lost motion receiver to secure the stylet in the trephine needle seating position when the trephine needle is being seated in the bone prior to boring.
11. The bone biopsy assembly of item 10, wherein the lost motion receiver includes an outer wall outlining a lost motion chamber adapted to accommodate sliding movement of the stylet during bone boring with the bone biopsy assembly.
12. The bone biopsy assembly of item 11, further including at least one window in the outer wall allowing one to view the stylet as it slides in the lost motion chamber of the lost motion receiver.
13. The bone biopsy assembly of item 12, further including indicia on (a) the trephine needle, (b) the lost motion receiver or (c) the trephine needle and the lost motion receiver whereby one is able to visually monitor a depth of the bone biopsy during bone boring.
14. The bone biopsy assembly of item 3, wherein the stylet includes an elongated body having a pointed tip at a first terminus and a head at a second terminus.
15. The bone biopsy assembly of item 14, further including a stylet seat at the second end of the trephine needle adapted for receiving and holding said head of said stylet when the stylet is positioned within the trephine needle in a trephine needle seating position with the pointed tip projecting beyond the cutting edge.
16. The bone biopsy assembly of item 15, wherein the stylet seat is in the form of an acircular socket and the head includes an acircular cross section whereby when the stylet is in the trephine needle seating position, the head is received in the acircular socket and the stylet is locked for rotation with the trephine needle.
17. The bone biopsy assembly of item 16, further including a removable stop that is engaged in an opening in the lost motion receiver to secure the stylet in the trephine needle seating position when the trephine needle is being seated in the bone prior to boring.
18. The bone biopsy assembly of item 1, wherein the lost motion receiver includes an outer wall outlining a lost motion chamber adapted to accommodate sliding movement of the stylet during bone boring with the bone biopsy assembly.
19. The bone biopsy assembly of item 18, further including at least one window in the outer wall allowing one to view the stylet as it slides in the lost motion chamber of the lost motion receiver.
20. The bone biopsy assembly of item 19, further including indicia on (a) the trephine needle, (b) the lost motion receiver or (c) the trephine needle and the lost motion receiver whereby one is able to visually monitor a depth of the bone biopsy during bone boring.
21. A method of procuring a bone core sample from a bone, comprising:
   engaging a pointed tip of a stylet into the bone to seat a cutting edge of a trephine needle in the bone;
   unlocking the stylet to allow free sliding movement of the stylet within a lumen of the trephine needle; and
   driving the trephine needle into the bone to procure the bone core sample within the lumen of the trephine needle while allowing the stylet to slide through the lumen of the trephine needle proximal to the bone core sample.
22. The method of item 21, including removing the trephine needle from the bone.
23. The method of item 22, including removing the stylet from the lumen of the trephine needle.
24. The method of item 23, including pushing the bone core sample out of the lumen of the trephine needle.
25. A method of procuring a bone core sample from a bone, comprising:
   connecting a driver to a bone biopsy assembly including (a) a lost motion receiver, (b) a trephine needle and (c) a stylet received for sliding movement within the lost motion receiver and a lumen of the trephine needle;
   engaging a pointed tip of the stylet into the bone to seat a cutting edge of a trephine needle in the bone;
   unlocking the stylet to allow free sliding movement of the stylet within the lumen of the trephine needle and the lost motion receiver; and
   driving the trephine needle into the bone to procure the bone core sample within the lumen of the trephine needle while allowing the stylet to slide through the lumen of the trephine needle in front of the bone core sample.
26. The method of item 25, including removing the trephine needle from the bone.
27. The method of item 26, including removing the stylet from the lumen of the trephine needle.
28. The method of item 27, pushing the bone core sample out of the lumen of the trephine needle.
29. The method of item 28, further including only connecting the driver to the bone biopsy assembly once during the procuring of the bone core sample.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified.

Although the bone biopsy assembly, the bone biopsy system and the method of procuring a bone core sample from a bone of this disclosure have been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

1-9. (canceled)

10. The bone biopsy assembly of claim 1, further including a removable stop that is engaged in an opening in the lost motion receiver to secure the stylet in the trephine needle seating position when the trephine needle is being seated in the bone prior to boring.

11. The bone biopsy assembly of claim 10, wherein the lost motion receiver includes an outer wall outlining a lost motion chamber adapted to accommodate sliding movement of the stylet during bone boring with the bone biopsy assembly.

12. The bone biopsy assembly of claim 11, further including at least one window in the outer wall allowing one to view the stylet as it slides in the lost motion chamber of the lost motion receiver.

13. The bone biopsy assembly of claim 12, further including indicia on (a) the trephine needle, (b) the lost motion receiver or (c) the trephine needle and the lost motion receiver whereby one is able to visually monitor a depth of the bone biopsy during bone boring.

14-16. (canceled)

17. The bone biopsy assembly of claim 1, further including a removable stop that is engaged in an opening in the lost motion receiver to secure the stylet in the trephine needle seating position when the trephine needle is being seated in the bone prior to boring.

18-24. (canceled)

25. A method of procuring a bone core sample from a bone, comprising:
connecting a driver to a bone biopsy assembly including (a) a lost motion receiver, (b) a trephine needle and (c) a stylet received for sliding movement within the lost motion receiver and a lumen of the trephine needle;
engaging a pointed tip of the stylet into the bone to seat a cutting edge of a trephine needle in the bone;
unlocking the stylet to allow free sliding movement of the stylet within the lumen of the trephine needle and the lost motion receiver; and
driving the trephine needle into the bone to procure the bone core sample within the lumen of the trephine needle while allowing the stylet to slide through the lumen of the trephine needle in front of the bone core sample.

26. The method of claim 25, including removing the trephine needle from the bone.

27. The method of claim 26, including removing the stylet from the lumen of the trephine needle.

28. The method of claim 27, pushing the bone core sample out of the lumen of the trephine needle.

29. The method of claim 28, further including only connecting the driver to the bone biopsy assembly once during the procuring of the bone core sample.

30. A bone biopsy assembly, comprising:
a lost motion receiver having a proximal end and a distal end;
a trephine needle including an elongated shaft having a first end, a second end and a lumen extending from the first end to the second end;
a stylet received for sliding movement within the lumen and the lost motion receiver wherein the stylet includes an elongated body having a pointed tip at a first terminus and a head at a second terminus;
a drive connector at the proximal end of the lost motion receiver and a cutting edge at the first end of the trephine needle;
a coupler releasably securing the lost motion receiver to the trephine needle wherein the coupler includes (a) a male connector at the second end of the trephine needle, (b) a cooperating female connector at the distal end of the lost motion receiver, (c) a locking ring carried on the lost motion receiver and (d) a cooperating locking boss carried on the trephine needle; and
a stylet seat at the second end of the trephine needle adapted for receiving and holding said head of said stylet when the stylet is positioned within the trephine needle in a trephine needle seating position with the pointed tip projecting beyond the cutting edge wherein the stylet seat is provided in the male connector and the stylet seat is in the form of an acircular socket and the head includes an acircular cross section whereby when the stylet is in the trephine needle seating position, the head is received in the acircular socket and the stylet is locked for rotation with the trephine needle.

31. A bone biopsy assembly, comprising:
a lost motion receiver having a proximal end and a distal end;
a trephine needle including an elongated shaft having a first end, a second end and a lumen extending from the first end to the second end;
a stylet received for sliding movement within the lumen and the lost motion receiver wherein the stylet includes an elongated body having a pointed tip at a first terminus and a head at a second terminus;
a drive connector at the proximal end of the lost motion receiver and a cutting edge at the first end of the trephine needle;
a coupler releasably securing the lost motion receiver to the trephine needle; and
a stylet seat at the second end of the trephine needle adapted for receiving and holding said head of said stylet when the stylet is positioned within the trephine needle in a trephine needle seating position with the pointed tip projecting beyond the cutting edge, wherein the stylet seat is in the form of an acircular socket and the head includes an acircular cross section whereby when the stylet is in the trephine needle seating position, the head is received in the acircular socket and the stylet is locked for rotation with the trephine needle.
